(19) United States
(12) Patent Application Publication
(10) Pub. No.: US 2012/0265008 A1
(43) Pub. Date: Oct. 18, 2012

(54) SHAFT FOR ENDOSCOPE WITH A RIGID SURGICAL SUCTION AND DETACHABLE RETRACTOR BLADE FOR ENDOSCOPIC MICRO-NEUROSURGERY OF THE BRAIN

(76) Inventors: SANDEEP SOOD, TROY, MI (US); BEENA GAIND SOOD, TROY, MI (US)

(21) Appl. No.: 13/442,812

(22) Filed: Apr. 9, 2012

Related U.S. Application Data
(60) Provisional application No. 61/517,109, filed on Apr. 13, 2011.

(51) Int. Cl. A61B 1/00

(52) U.S. Cl. 600/104

(57) ABSTRACT

A shaft for endoscope for two handed micro neurosurgery of the brain comprising a Rigid surgical suction at 6 o′clock position that is longer than the shaft, Channel for endoscope at 12 o′clock position, Channel for irrigation, Surgical suction control hole on the shaft, a hand rest on the shaft and a detachable retractor blade.

### CROSS REFERENCE TO RELATED APPLICATIONS

The application claims the benefit of priority of U.S. Provisional Application No. 61/517,109, filed Apr. 13, 2011, entitled “A shaft for endoscope with a rigid surgical suction and detachable retractor blade for endoscopic micro-neurosurgery of the brain”, the disclosure of which is expressly incorporated herein by reference in its entirety.

### STATEMENT REGARDING FEDERALLY SPONSORED RESEARCH OR DEVELOPMENT

Not Applicable

### DESCRIPTION OF ATTACHED APPENDIX

Not Applicable

### BACKGROUND OF THE INVENTION

This invention relates generally to the field of endoscopic brain surgery and more specifically to a shaft for endoscope with rigid surgical suction and detachable retractor blade for two handed dissection surgery. The invention is designed to improve micro-neurosurgery of the brain.

Surgery of the brain is done with a microscope that provides visualization and magnification. The microscope is generally fixed in one position and to move to the area of interest the surgeon physically moves the microscope head holding the handles provided for this purpose. Once the surgeon is satisfied with the position of the microscope, he generally performs the surgery looking through the microscope at the tissue using one instrument in one hand and the other instrument in the other hand. Generally a surgical suction with low pressure is used in the left hand to keep the field clear of blood. A second instrument is used in the right hand. This may be a dissecting instrument, cutting instrument or any of the myriad of instruments that are used to manipulate tissue as deemed necessary by the surgeon. The two instruments, surgical suction in the left hand and the instrument in the right hand are used in conjunction with each other. The surgical suction in-addition to keeping the field clear of blood is also used to provide counter pressure on the tissue to aid dissection with the second instrument in the right hand. This type of two handed or bimanual ability to manipulate the tissue with two instruments is critical to the objective of surgery which is to dissect and separate abnormal tissue from normal tissue. Bleeding during surgery is controlled with an instrument called bipolar cautery foreceps, which is used when needed by replacing the instrument in right hand. During the course of surgery the surgeon may need to move the position of the microscope head numerous times to get optimal visualization. He may accomplish this by handing instrument in the right hand to the assistant and using the handle on the microscope to move it to a new position and then resume the surgery once satisfied with the new position of the microscope. Small retractor blades may be used to push tissue to one side so as to provide a path through which surgery can be done in the deeper part of the brain.

Some surgeries of the brain may be accomplished using a minimally invasive surgical approach. In these procedures the visualization and magnification is provided by an endoscope. Since the endoscope is much smaller, the incision required for the operation is also smaller, hence called minimally invasive. Endoscope also has an advantage to provide better illumination and magnification compared to a microscope. Surgery is done with an endoscope in two ways. In the first way, through a small incision, the endoscope is introduced into the brain where surgery is to be performed. In the prior art, the endoscope is generally fixed to a holder. An instrument is passed through a co-axial instrument channel present in the endoscope shaft. Generally the instrument is a flexible jaw that can be opened and closed or scissors that can be used for cutting. However, only one instrument can be introduced at one time. Manipulation with the instrument is limited to pulling or cutting since the instrument can be moved only in and out and not sideways to allow for dissection. Sometimes the surgeon may hold the endoscope in the left hand and introduce the instrument into the endoscope channel through the right hand to accomplish surgery. Since there is only one instrument in the surgical field bimanual or two handed fine dissection is not possible.

In the second way of using the endoscope to accomplish brain surgery, a large port about 1-1.5 cm diameter is passed through the brain to provide a path for operation. This is similar to using small retractor blades to provide a path to the area of interest when surgery is accomplished with a microscope. The endoscope is then fixed to the holder and passed through the port to the area of interest. Through the rest of the space in the port, two instruments are passed, one held in the left hand and the other in the right hand and tissue dissection is accomplished in a two handed bimanual fashion.

The prior art of endoscopic surgery on the brain as described above is plagued with major problems.

Endoscopic brain surgery in the first way has major problems. First, there is only one instrument channel and the instruments can be moved only in one axis, i.e. in and out. Tissue dissection to accomplish surgery in this manner is significantly more difficult and time consuming. Second, the position of the endoscope is fixed by the holder. As a result to move to a different area of interest the surgeon has to stop, release the endoscope from the holder and reposition the endoscope. This is not only frustrating but also tiring and time consuming. Thirdly, only a small flexible suction can be passed through the instrument channel to accomplish removal of debris and blood. This can be done only intermittently since the channel is also used for other instrument used in surgery. This makes it inefficient and increases the duration of surgery and is time consuming for the surgeon. Further since the suction is flexible it cannot provide enough counter pressure to aid in tissue dissection.

Endoscopic brain surgery in the second way has problems as well. First, a large part of the space in the port is occupied by the endoscope. Using two instruments through the rest of the narrow space in the port to accomplish dissection at a depth is difficult and tedious. It results in scissoring of instruments. Second, since the endoscope is fixed to a holder the surgeon has to constantly stop and reposition the endoscope to visualize the area of interest. This increases the duration of surgery, tires the surgeon and increases the risk of the operation.

Finally, in prior art, the endoscope accumulates debris and blood on the lens from the splatter. It has to be repeatedly removed from the field of surgery and cleaned. This is time consuming and frustrating for the surgeon.

Komiya U.S. Pat. No. 4,043,323 described a medical instrument attached to an endoscope. However this device does not provide any means of suction or irrigation.

Heckele U.S. Pat. No. 4,881,523 describes an endoscope for endonasal surgery comprising a suction and a flushing shaft. The suction is contained within the shaft and does not protrude beyond and hence cannot be used for bimanual dissection.

Arias U.S. Pat. No. 5,429,596 describe a similar device in which an electrocautery can be inserted through a side channel.

McFarlin U.S. Pat. No. 5,667,478 discloses a fiberoptic scope that can be attached to a medical instrument. Such does not provide a means for irrigation or suction. Further a fiberoptic scope has poor image resolution.

Iafrati U.S. Pat. No. 6,129,661 describes an endoscopic instrumentation with a working channel for use in subfascial endoscopic surgery. This instrumentation cannot be used for bimanual dissection.

Rudischhauser U.S. Pat. No. 6,471,639 discloses a rigid shaft with multiple channels for the endoscope, instrumentation, light and irrigation or suction. The suction channel is contained within the shaft and does not protrude beyond the shaft limiting its usefulness in bimanual dissection

Kent U.S. Pat. No. 6,585,642 discloses a flexible endoscope with a removable suction tube. Such flexible endoscopes cannot be used for bimanual dissection instrumentation.

It is the intent of the present invention to allow use of endoscope and yet permit two handed tissue dissection, ability to constantly suction debris and blood from the area of interest and permit surgery without need for constantly releasing the endoscope from the holder to reposition it to the area of interest while retaining the advantages of endoscopic surgery that is, small incision, better illumination and higher magnification.

### BRIEF SUMMARY OF THE INVENTION

The primary object of the invention is to be able to do two handed dissection of tissues during endoscopic brain surgery.

Another object of the invention is to constantly suction away debris and blood from the field of surgery.

Another object of the invention is to be able to control the strength of the surgical suction

Another object of the invention is to be able to constantly irrigate the surgical field.

A further object of the invention is to prevent the splatter from tissue dissection on the lens of the endoscope

Another object of the invention is to be able to constantly position the endoscope to the area of interest without cessation in surgery

Another object of the invention is to stabilize the endoscope with a hand rest on the shaft

Another object of the invention is to be able to retract the brain tissue with a retractor blade that can be attached to the endoscope shaft

Other objects and advantages of the present invention will become apparent from the following descriptions, taken in connection with the accompanying drawings, wherein, by way of illustration and example, an embodiment of the present invention is disclosed.

In accordance with a preferred embodiment of the invention, there is disclosed a shaft for endoscope for two handed surgical dissection during endoscopic brain surgery comprising: A Rigid surgical suction at 6 o′clock position, Channel for endoscope at 12 o′clock position, Channel for irrigation adjacent to the surgical suction, a suction control hole on the shaft and a detachable retractor blade.

### BRIEF DESCRIPTION OF THE DRAWINGS

The drawings constitute a part of this specification and include exemplary embodiments to the invention, which may be embodied in various forms. It is to be understood that in some instances various aspects of the invention may be shown exaggerated or enlarged to facilitate an understanding of the invention.

FIG. 1A shows the prior art of brain surgery with a surgical microscope

FIG. 1B shows prior art of brain surgery with an endoscope

FIG. 1C show prior art of brain surgery using a port with an endoscope fixed in position

FIG. 2A shows the present invention with a rigid suction that is longer than the shaft

FIG. 2B shows an alternative embodiment of the invention with a changeable surgical suction tip

FIG. 3 shows the modality of use of the present invention with a port

FIG. 4 show the alternate way of using the device with the attached retractor blade without a port.

### DETAILED DESCRIPTION OF THE PREFERRED EMBODIMENTS

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

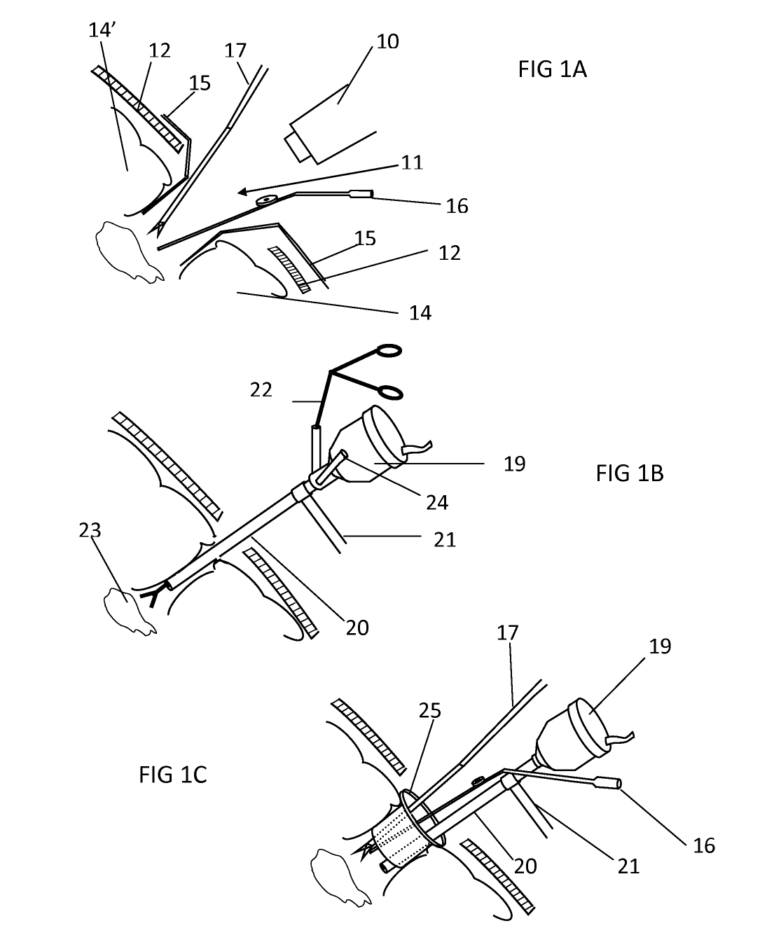

FIG. 1A shows the prior art of brain surgery with a microscope 10. An opening, 11 in the bone 12 allows tissue 14 & 14′ to be spread apart with a retractor blade 15. The surgeon operates holding two instruments 16, generally a surgical suction, and 17, generally a forceps, dissecting instrument, scissor or any of the myriad instruments suited for the procedure being performed. The surgical suction keeps the field clear of blood and debris and in conjunction with the second instrument is used for dissecting tissue planes in a two handed bimanual fashion.

FIG. 1B shows prior art of brain surgery with an endoscope 19, that passes through a shaft 20 at 6 o′clock position. It may be fixed to a holder 21. The coaxial instrument channel, generally at 12 o′clock position, is used to push an instrument 22 with jaws or scissors into the area of interest and tissue 23 that is to be removed or repaired. Bi-manual dissection of the tissue is difficult since there is a single instrument that can move in only one plane, that is, in and out. Some endoscopes may have additional co-axial channel 24 for using a flexible suction or irrigation to remove debris and blood.

FIG. 1C show prior art of brain surgery using a port 25 placed through the brain tissue to the area of interest. The endoscope 19 is fixed to a holder 21 at the shaft 20. Two instruments 16 & 17 are then used through the limited space in the port to accomplish surgery. The shaft 20 is released from the holder 21 and re-positioned every time the area of interest changes, to obtain a better view of the operative field. Endoscope 19 may need to be removed from the shaft periodically and cleaned if the lens accumulates debris or blood and obscures vision.

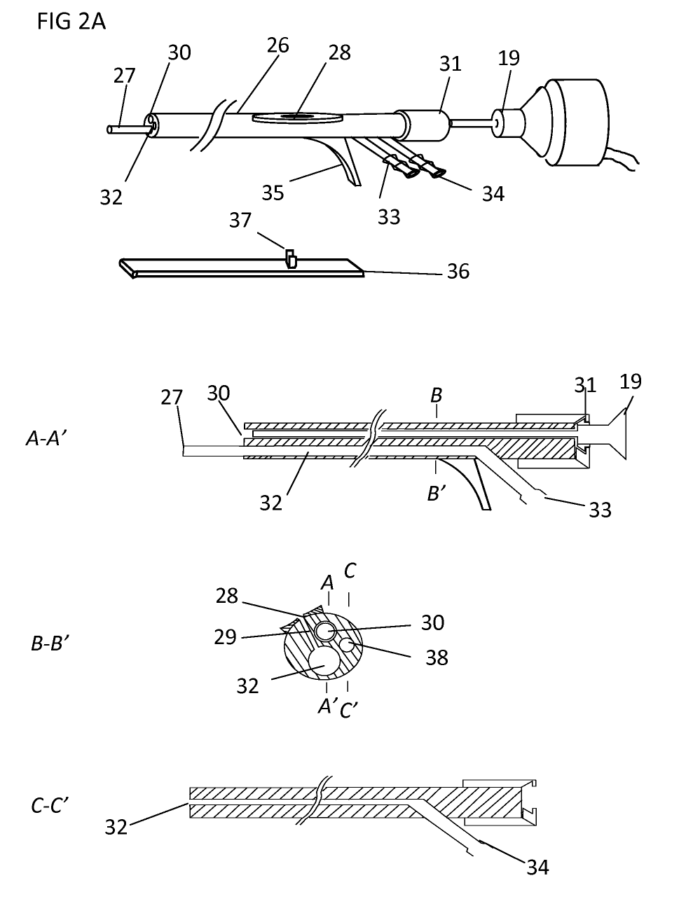

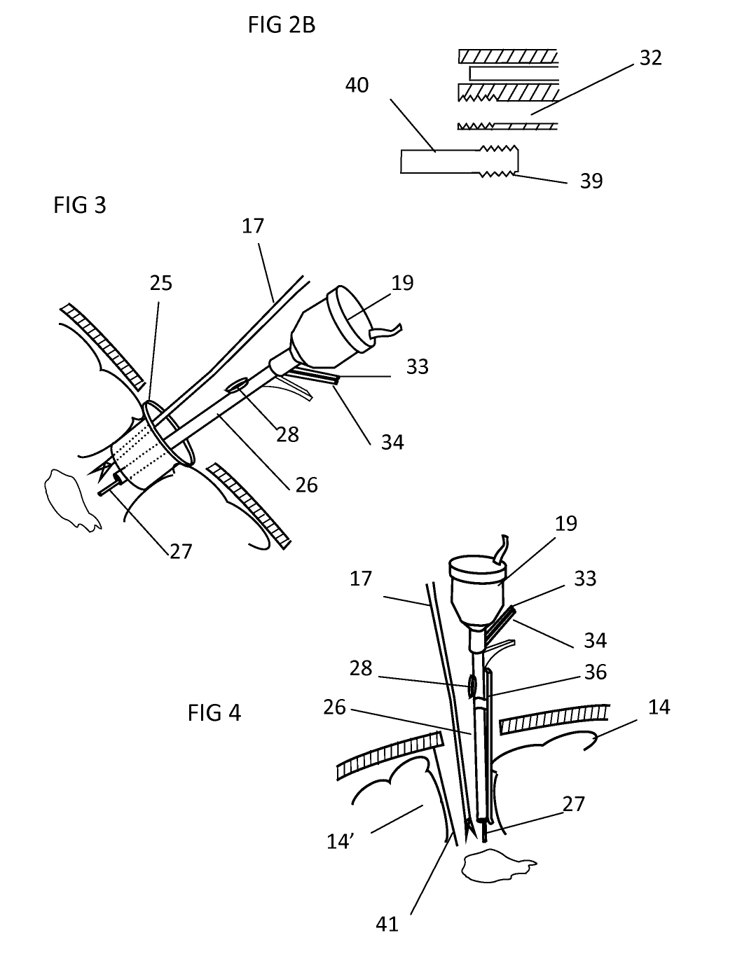

FIG. 2A shows the present invention comprising a shaft 26 containing a rigid surgical suction 27 at 6 O′clock position. The suction has a control hole 28 connected to the suction channel by a channel 29 in or around the shaft 26. Channel 30 at 12 O′clock position is for the endoscope 19 that locks to the shaft through a lock 31. In the preferred embodiment the channel 30 is longer than the endoscope but shorter than the surgical suction. The surgical suction runs in the channel 32. Additional irrigation channels 38 at positions about 3 or 9 O′clock extends through the entire length of the shaft to the tip. Attachment 33 & 34 connect tubing for irrigation and suction respectively. There is a hand support 35 that rest between the thumb and forefinger of the surgeon in an ergonomic fashion. A retractor blade 36 can be attached to the shaft 26 with the snap 37.

FIG. 2B shows an alternative embodiment where different sizes of the surgical suction tip 40 can be interchangeably attached to the channel 32 with any of the varied means such a thread screw 39.

FIG. 3 shows the modalities of use of the present invention. The device is introduced through a port 25 and is held in the left hand. A second instrument 17 is used through the port in the right hand. Under visualization provided by the endoscope 19, the suction 27 is used in conjunction with instrument 17 to do two handed bimanual dissection of the tissues. The suction 27 constantly keep the field of surgery clear of blood and debris and it strength is modulated by the degree to which the hole 28 is covered by the thumb of the hand holding the shaft 26. Irrigation fluid may be run continuously or intermittently through the 33 and suction through 34. The channel 30 for the endoscope being longer than the endoscope itself prevents and minimizes debris and blood splattering on to the lens of the endoscope and obscuring vision. As the surgeon moves the suction to the field of interest by moving the shaft 26 held by the left hand, the endoscope by virtue of being in the shaft is also directed toward that area of interest obviating the need for constant releasing it from the holder and repositioning it as in the prior art.

FIG. 4 show the alternate way of using the device without a port or a retractor. The device is held in the left hand and slipped under the surface of the brain or between the two hemispheres of the brain. The shaft 26 has a retractor blade 36 attached to it that provides retraction to one side brain 14 whereas the other side of the brain 14′ is kept in place by a rigid anatomical structure 41 called Falx. A second instrument in 17 held in the right hand is then able to accomplish surgery in conjunction with suction 27 in the device in a two handed bimanual fashion.

While the invention has been described in connection with a preferred embodiment, it is not intended to limit the scope of the invention to the particular form set forth, but on the contrary, it is intended to cover such alternatives, modifications, and equivalents as may be included within the spirit and scope of the invention as defined by the appended claims.

What is claimed is:

1. A shaft for endoscopic micro-neurosurgery comprising:
Rigid suction at 6 o′clock position;
Channel for endoscope at 12 o′clock position;
Channel for irrigation adjacent to the suction;
Surgical suction control hole on the shaft
Hand rest on the shaft
Attachable retractor blade

2. A shaft for endoscopic micro-neurosurgery in claim 1, which has a rigid surgical suction that is longer than the shaft

3. A shaft for endoscopic micro-neurosurgery in claim 1, which has a channel for endoscope that is a longer than the endoscope.

4. A shaft for endoscopic micro-neurosurgery in claim 1, which has a surgical suction tip may be removable and available in different lengths for attachment

5. A shaft for endoscopic micro-neurosurgery in claim 1, which has retractor blade that may be attached to the shaft

6. A shaft for endoscopic micro-neurosurgery in claim 1, which has retractor blades available in different lengths and widths

7. A shaft for endoscopic micro-neurosurgery in claim 1, which has a hand rest to support and stabilize the system
